(19) United States
(12) Patent Application Publication
(10) Pub. No.: US 2024/0285340 A1
(43) Pub. Date: Aug. 29, 2024

(54) METHOD FOR MONITORING MICROWAVE ABLATION STATUS

(71) Applicants: Kansas State University Research Foundation, Manhattan, KS (US); Precision Microwave, Inc., Manhattan, KS (US)

(72) Inventors: Punit Prakash, Manhattan, KS (US); Hojjatollah Fallahi, Manhattan, KS (US); Austin Pfannenstiel, Manhattan, KS (US)

(21) Appl. No.: 18/572,857

(22) PCT Filed: Jun. 21, 2022
(86) PCT No.: PCT/US2022/034357

§ 371 Date: Dec. 21, 2023

Related U.S. Application Data
(60) Provisional application No. 63/213,232, filed on Jun. 22, 2021.

(51) Int. Cl. A61B 18/18; A61B 18/00

(52) U.S. Cl. CPC A61B 18/1815; A61B 2018/00577; A61B 2018/00654; A61B 2018/00708; A61B 2018/00779; A61B 2018/00875; A61B 2018/1823

(57) ABSTRACT

A method of ablating tissue uses a microwave ablation system with a plurality of applicators. The method includes the steps of having the applicators inserted into the tissue and located in and/or adjacent a target tissue section and having the system perform an ablation cycle by activating at least one of the applicators so that the at least one applicator emits electromagnetic radiation that is sufficiently strong to cause ablation of the target tissue section. The at least one applicator comprises a directional applicator that emits radiation to define an angular radiation pattern. The step of having the applicators inserted into the tissue includes the step of orienting the directional applicator so that the angular radiation pattern extends from the directional applicator toward the target tissue section. The method also includes the step of having the system perform a sensing cycle that includes the step of using one or more of the applicators to take a measurement associated with a dielectric property of the tissue.

### RELATED APPLICATION

This application claims the benefit of U.S. Provisional Patent Application No. 63/213,232, filed Jun. 22, 2021, which is incorporated by reference herein in its entirety.

### STATEMENT REGARDING FEDERALLY SPONSORED RESEARCH OR DEVELOPMENT

This invention was made with government support under grant number 1819177 awarded by the National Science Foundation (NSF). The government has certain rights in the invention.

### BACKGROUND

### 1. Field

The present invention relates generally to systems and methods for tissue ablation. More specifically, embodiments of the present invention concern systems and methods for transmitting, receiving, and analyzing electromagnetic signals between ablation applicators and though a target tissue, such as a tumor, with the purpose of determining the biophysical state of the target tissue and providing the clinical user an indication whether the target tissue has been adequately treated during a thermal ablation procedure.

### 2. Discussion of Prior Art

Image-guided thermal ablation is a minimally-invasive treatment for tumors in the liver, kidney, lung and other tissues, typically for patients who are not good surgical, radiotherapy, or chemotherapy candidates. The goal of thermal ablation procedures is to deliver a lethal thermal dose to the target, which consists of the tumor and a 5-10 mm surrounding margin. While several energy modalities, including cryoablation, radiofrequency ablation (RFA), microwave ablation (MWA), and laser have been applied clinically, MWA has several key advantages over other ablation technologies such as faster ablation time, ability to produce larger ablation zones, and less need for additional system components such as grounding pads. During MWA, an antenna is inserted into, or placed in close proximity to, the target tissue and radiates electromagnetic energy at microwave frequencies that causes frictional heating of the surrounding tissue as polar molecules in the tissue, such as water, attempt to align with the electromagnetic field. Thermal damage induced by ablation is a function of the time and temperature history of the tissue; irreversible but non-lethal damage may occur above 42° C. while near-instant tissue death occurs at temperatures of 60° C. Although MWA provides a minimally invasive, non-toxic, low cost, outpatient therapy that has comparable outcomes to the gold standard surgical resection for treatment of small tumors, local recurrence rates remain considerably higher for larger tumors (>3 cm diameter). Inadequate and inaccurate thermal dose delivery to the targeted tumor leading to failure of adequate treatment margins are believed to be the most significant causes of thermal ablation's higher local recurrence rates, compared to surgical resection or radiotherapy.

Several patient-specific and procedural factors contribute to inadequate thermal dose delivery. Tissue biophysical properties (primarily electromagnetic and blood perfusion) have a considerable impact on microwave absorption and bioheat transfer, and ultimately ablation zone size and shape. These properties are a function of the physical and pathological state of the tumor and background tissue (i.e. vascular and cellular density, underlying presence of steatosis, calcification, cirrhosis, and fibrosis) and dynamically change during ablation procedures as a function of tissue temperature and hydration. However, current ablation systems do not provide any means to assess tissue biophysical state and optimize energy delivery based on characteristics of an individual patient's disease; rather, ablative dose is selected based on a simplified assessment of basic anatomic/geometric considerations, such as tumor size and shape, and proximity to critical structures. Furthermore, directly observing the growth of the thermal ablation zone in real time during the procedure is not possible with conventional medical imaging techniques. Applying excessive power for longer duration than necessary will typically risk unpredictable growth of the thermal ablation zone beyond the target to cause damage to nearby healthy sensitive and critical anatomy that may cause pain or medical complications, which in severe cases could be life-threatening. Additionally, there is growing evidence indicating that sub-ablative temperatures within normal tissue resulting from prolonged ablation procedures may have pro-tumorigenic effects, further underscoring the need to monitor and promptly terminate ablations. Consequently, the success of ablation procedures is highly dependent on clinician skill and experience to balance the consequences of collateral damage for over-treatment or disease recurrence for under-treatment.

Currently, adequate dose delivery to the target tissue is determined on post-treatment contrast-enhanced imaging (typically, CT or ultrasound), with the ablated region presenting as non-enhancing, nonviable tissue. The time at which follow-up imaging is performed varies across institutions, ranging from immediately post-procedure to a few weeks post-procedure. Even when immediate post-ablation imaging is performed, the ablation applicators are often withdrawn to reduce imaging artifacts, and indication of incomplete ablation would require complete reperformance of the procedure, including applicator repositioning. This extra time can place severe burdens on hospitals where the CT-suite is a capacity constrained resource. Furthermore, it can be technically challenging to determine spatial overlap of the ablation zone observed post-procedure with pre-treatment tumor imaging, due to considerable contraction and deformation of tissue during ablative procedures. Finally, as the administration of contrast agent must be limited to minimize the risk of adverse effects to the patient, doctors must often make a difficult choice of administering contrast pre-procedure to more accurately position the ablation applicator in the tumor or wait until post-procedure to have more accurate confirmation of complete ablation of the target.

Previously, all microwave ablation applicators were configured to radiate in cylindrically symmetric patterns producing ellipsoidal or spherical ablation zones centered on the applicator axis. To treat relatively large tumors, clinicians commonly utilize multiple MWA applicators to bracket tumors and attempt treatment with a composite ablation zone. The use of multiple cylindrically-symmetric applicators therefore risks unintended damage to a substantial amount of healthy tissue surrounding the target tumor when trying to achieve a conformal ablation zone.

Additionally, past literature has described attempts to use changes in an ablation applicator's impedance matching (s₁₁) during an ablation procedure to infer tissue biophysical state and provide indication of the status of the ablation. However, these techniques are largely incapable of managing patient-specific variability and cannot accurately resolve changes in the target tumor from those of the surrounding healthy tissue. Moreover, the s₁₁ parameter is most sensitive to tissue in close proximity to the applicator and may not be well suited to detecting changes at the periphery of the tumor region/ablation zone boundary, which is the most critical region for ensuring treatment success. Further attempts to use low frequency impedance or electromagnetic transmission loss (s₂₁) between multiple ablation applicators or ablation applicators and other sensors further fail to focus only on the target tissue and are these systems also influenced by surrounding healthy tissue which reduces system sensitivity to an unusable level for clinical use. Of note, low frequency systems using electrode impedance tomography/spectroscopy require a substantial number of electrodes to encompass the target zone.

Recently, directional microwave ablation applicators, such as those described in U.S. Patent Application Publication Nos. 2017/0265940 and 2020/0367966, offer the ability to control the energy deposition pattern along the angular expanse through the use of reflectors, shields, and/or window structures. These directional applicators offer the ability to precisely target the electromagnetic radiation toward the target tissue and away from the non-target healthy surrounding tissue.

Using multiple directional applicators positioned along the target periphery, enables an “outside-in” ablation technique that could ensure the necessary treatment margin is achieved. An “outside-in” approach also provides the unique opportunity to use the ablation applicators both as therapy delivery and monitoring devices to detect electromagnetic characteristics of intervening target tissue. The sensed electromagnetic changes in the target tissue can be used to infer the ablative state of the target and provide an indication of when complete ablation of the target has been achieved. The current invention could reduce the skill and experience necessary and the significant burden placed on individual clinicians to execute an effective ablation procedure. Clinical adoption of the current invention could provide for precise and personalized delivery of thermal tumor ablation and expand access to low-cost, minimally invasive, outpatient cancer treatment, reducing the burden on patients, their families, and the health system.

This background discussion is intended to provide information related to the present invention which is not necessarily prior art.

### SUMMARY

The following brief summary is provided to indicate the nature of the subject matter disclosed herein. While certain aspects of the present invention are described below, the summary is not intended to limit the scope of the present invention.

Embodiments of the present invention provide a method of ablating tissue that does not suffer from the problems and limitations of the prior art methods, including prior art methods set forth above.

A first embodiment of the present invention concerns a method of ablating tissue using a microwave ablation system with a plurality of applicators. The method broadly includes the steps of having the applicators inserted into the tissue and located in and/or adjacent a target tissue section and having the system perform an ablation cycle by activating at least one of the applicators so that the at least one applicator emits electromagnetic radiation that is sufficiently strong to cause ablation of the target tissue section. The at least one applicator comprises a directional applicator that emits radiation to define an angular radiation pattern. The step of having the applicators inserted into the tissue includes the step of orienting the directional applicator so that the angular radiation pattern extends from the directional applicator toward the target tissue section. The method also broadly includes the step of having the system perform a sensing cycle that includes the step of using one or more of the applicators to take a measurement associated with a dielectric property of the tissue.

A second embodiment of the present invention concerns a microwave ablation system configured to ablate tissue. The microwave ablation system broadly includes a microwave source, a plurality of applicators, a metering device, and an optional switching device. The applicators are operable to be inserted into the tissue and located in and/or adjacent a target tissue section. At least one of the applicators is configured to receive power from the microwave source during an ablation cycle and emit electromagnetic radiation that is sufficiently strong to cause ablation of the target tissue section. The at least one applicator comprises a directional applicator that emits radiation to define an angular radiation pattern, with the directional applicator configured to be oriented so that the angular radiation pattern extends from the directional applicator toward the target tissue section. The metering device is configured to receive a signal from at least one of the applicators during a sensing cycle to take a measurement associated with a dielectric property of the tissue. The optional switching device is operably coupled to the applicators and is configured to be selectively coupled to the microwave source and/or the metering device to switch the system between the ablation cycle and the sensing cycle.

This summary is provided to introduce a selection of concepts in a simplified form that are further described below in the detailed description. This summary is not intended to identify key features or essential features of the claimed subject matter, nor is it intended to be used to limit the scope of the claimed subject matter. Other aspects and advantages of the present invention will be apparent from the following detailed description of the embodiments and the accompanying drawing figures.

### BRIEF DESCRIPTION OF THE DRAWING FIGURES

Preferred embodiments of the invention are described in detail below with reference to the attached drawing figures, wherein:

FIG. 1 is a schematic illustration showing how tissue relative permittivity and the transmission coefficient between two ablation applicators changes during a thermal ablation procedure;

FIG. 2 is a schematic illustration showing an example broadband transmission coefficient between two MWA applicators pre-ablation and post-ablation;

FIG. 3 is a schematic illustration of an intra-procedural feedbacks system using a switching network, a vector network analyzer, and a pair of directional applicators;

FIG. 4 is a schematic illustration of the pathways the transmission coefficient could be measured between three directional applicators;

FIGS. 5A and 5B are schematic illustrations of the transmission coefficient measurements changing during the course of a thermal ablation;

FIGS. 5C-5F are photographs of tissue ablation progression over 2 min, 3 min, 4 min, and 10 min, respectively;

FIG. 6 is a schematic illustration of an intra-procedural feedbacks system using a switching network and a power meter;

FIG. 7 is a schematic illustration of an intra-procedural feedbacks system using a multiple independent microwave power sources and multiple power meters; and

FIG. 8 includes an illustration of incomplete ablation of intervening tissue and another illustration of complete ablation of intervening tissue.

FIGS. 9A and 9B depict raw average transmission coefficient and group delay curves during an exemplary ablation procedure.

FIGS. 9C and 9D depict normalized, time differentiated average transmission coefficient and group delay curves.

The drawing figures do not limit the present invention to the specific embodiments disclosed and described herein. The drawings are not necessarily to scale, emphasis instead being placed upon clearly illustrating the principles of the preferred embodiment.

### DETAILED DESCRIPTION OF THE PREFERRED EMBODIMENTS

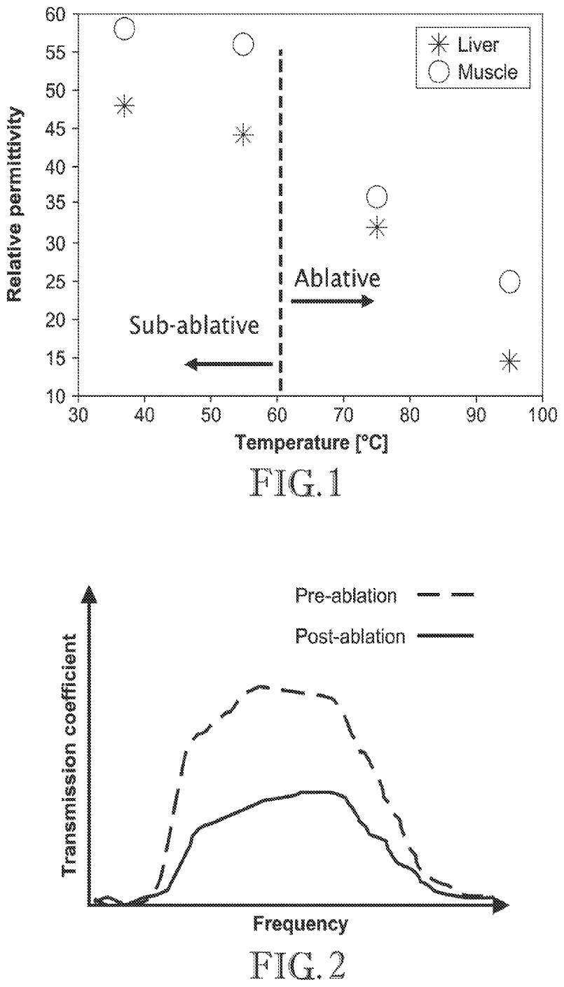

The dielectric properties of tissue (relative permittivity and conductivity) are known to change substantially at elevated temperatures due to dehydration and protein denaturation. FIG. 1 illustrates this dielectric contrast between tissue at ablative and sub-ablative temperatures for bovine liver and muscle at microwave frequencies. Although no temperature-dependent dielectric properties have been reported for tumors at microwave frequencies, a similar drop in dielectric properties at ablative temperatures has been reported at lower frequencies for excised human metastatic liver tumors. While the majority of these data have been measured in freshly excised ex vivo tissue, it is anticipated that similar changes occur in the in vivo setting where tissue is perfused. In addition to tissue desiccation, at ablative temperatures blood perfusion diminishes due to microvascular stasis, providing possibly even greater dielectric contrast between ablated and unablated tissue, as has been shown during liver RF ablation in vivo.

Antenna transmission coefficient, which is the fraction of power radiated by one antenna that is intercepted by another antenna, is a function of antenna geometry and dielectric properties of media in the channel between the transmitting and receiving antenna. During microwave ablation (MWA), the antenna geometry is fixed and unchanging inside the applicator, but the transmission coefficient between the applicators changes as the intervening tissue is heated and desiccated. FIG. 2 shows an example of the pre-ablation and post-ablation transmission coefficient between two microwave ablation applicators (MWA applicators). Therefore, changes in transmission coefficients between multiple MWA applicators can be directly mapped to changes in tissue electromagnetic properties at elevated temperatures and provide a means to monitor tissue state during “outside-in” ablation with multiple MWA applicators. When the real-time changes in transmission coefficient correspond to adequate thermal ablation of the intervening target tissue, then the present invention provides an input that could automatically terminate the ablation procedure or serve as an operator aid to the clinical user to suggest they should terminate the ablation procedure manually.

If omni-directional ablation applicators are used to bracket a target tissue, then the resulting transmission coefficient between those applicators will be sensitive to both the dielectric properties of the intervening target tissue as well as the surrounding non-target healthy tissue. This surrounding non-target tissue may be comprised of heterogenous material, such as different tissue types, other organs, blood vessels, or other anatomical structures, each of which may experience changes in tissue dielectric properties, in different or unpredictable ways, during thermal ablation. For example, large blood vessels, which act as a heat sink, may cool the surrounding tissue and minimize the changes in the dielectric properties of that tissue that may otherwise be expected during thermal ablation. As such, surrounding non-target tissues and anatomical structures influence the transmission coefficient between ablation applicators in unpredictable ways and should be excluded in order to use the transmission coefficient between ablation applicators as a reliable indication of complete ablation of only the desired target tissue. Recently developed directional microwave ablation antennas have a radiation pattern where the −10 dB sensitivity range extends over less than one-third of the angular expanse. Therefore, when using directional ablation applicators to bracket a target tissue, the relative signal level transmitted between applicators would be especially sensitive to the target tissue, but also especially in-sensitive to adjacent non-target heterogenous healthy tissue. The relative transmitted signal can be assessed with a complex-valued (i.e. magnitude and phase) transmission coefficient, which is the ratio of received signal to the transmitted signal. Alternatively, the transmission coefficient could be a real-valued quantity, determined from the ratio of the power of the signal received to the power of the signal transmitted. Alternatively, a measure of the relative signal transmitted may be assessed by the difference between the received and transmitted signal. The transmission coefficient between two directional ablation applicators can therefore give an indication of the changes in dielectric properties of only the intervening target tissue and could provide a reliable indication of complete ablation of the target. Other measures that consider the received signal relative to the transmitted signal would also apply. We use the term transmission coefficient generally in this application to refer to the relative level of the received signal to the transmitted signal (thus capturing ratios, differences, or other comparisons).

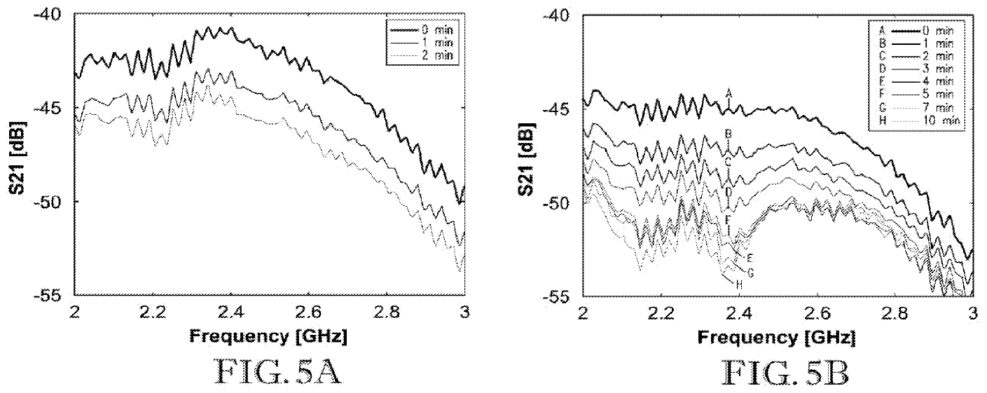

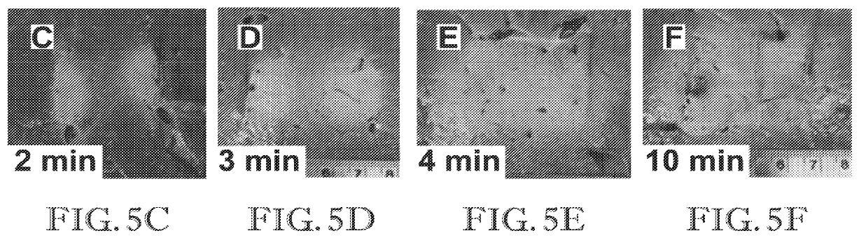

In one embodiment, when operating a pair of directional antennas in the sensing mode, the transmission coefficient over a range of frequencies can be measured. The frequency range of interest is generally around the operating frequency of the antennas used. For instance, antennas operating at 2.45 GHz, the frequency range of interest can be from 1 to 3 GHz, 2 to 3 GHZ, or 2.2 to 2.7 GHZ. Because transmission coefficient can vary based upon frequency, see FIGS. 5A and 5B particularly, it may be useful to process this data into a simpler form that can be used to provide feedback related to the progress of tissue ablation. The transmission coefficient data can be used to compute an average transmission coefficient (ATC) and/or a group delay (GD) value.

The ATC “collapses” the transmission coefficient data into a single value according to the following formula:

$$A T C = \frac{1}{N} {\sum }_{i = 1}^{N} \vert {s}_{21} ( {f}_{i} ) \vert$$

where f is frequency and fᵢ is a frequency within the sensing frequency range. In essence, ATC is an average of the magnitude of the transmission coefficient at multiple frequencies.

GD can be computed as

$$G D = - \frac{d ϕ ( {s}_{21} )}{d ω}$$

where ϕ is the phase and ω is the angular frequency.

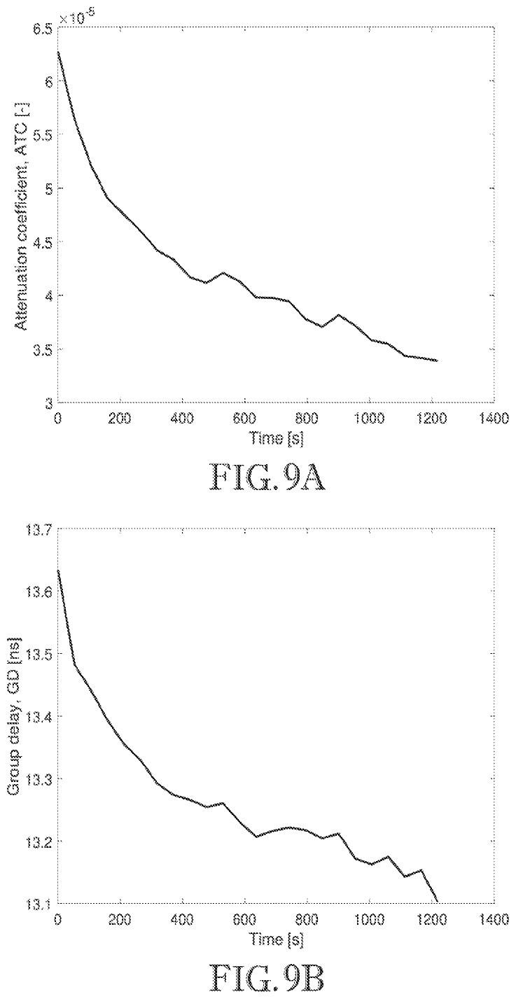

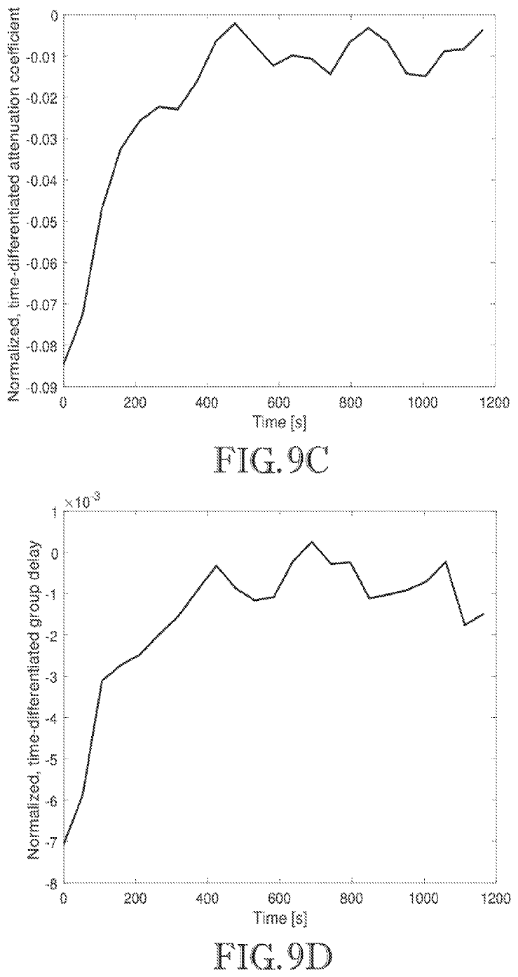

It has been discovered that both ATC and GD trend downwards and then saturate over the course of the ablation. These trends are depicted in FIGS. 9A and 9B. It was also discovered that for unknown reasons the initial value of the ATC and GD could vary across multiple ablation operations. Thus, in certain embodiments, it may be preferable to normalize ATC and GD against baseline values, such as a baseline average transmission coefficient and a baseline group delay, to allow for more ready comparison of values computed from different ablation operations. FIGS. 9C and 9D depict normalized, time-differentiated curves for ATC and GD. As the time differentiated curves approach zero, it is an indication that a saturation point has been achieved and ablation is complete.

Although directional MWA applicators provide an inherent advantage in improved sensitivity to the target tissue, for at least certain aspects of the invention, some combination of non-directional and directional applicators, or non-directional only applicators may be used depending on the specific configuration of the ablation system. The use of omni-directional ablation applicators with the current invention may be possible if transmission coefficient data developed through directional ablation can be further processed by advanced data analysis techniques to identify key indications that could not be discerned without a substantial volume of highly sensitive clinical data.

One of the advantages of MWA over other thermal therapy energy modalities is that the MWA heating process can quickly achieve very high (>100° C.) central peak temperatures. The high peak temperatures establish a steep thermal gradient that helps grow and expand the size of the ablation zone through thermal conduction. With conventional cylindrically-symmetric applicators, the thermal gradient expands the ablation zone in all directions and enables MWA to create larger treatment zones than competing energy modalities. However, for directional MWA (DMWA) applicators that are designed to radiate microwave energy in a preferred direction and develop directional ablation zones, excessive thermal conduction can be counterproductive. In this case, heat transfer through thermally conductive biological tissues can detrimentally expand the ablation zone into the non-target direction, causing an overall reduction in the directivity ratio (defined as the forward ablation depth divided by the backward ablation depth). When considering the anticipated clinical use of DMWA to direct heat towards a target tumor while avoiding thermal injury to nearby critical anatomy, the necessity of limiting undesired thermal conduction in the backward direction is evident. Reducing the applied power level would reduce the magnitude of the peak temperature driving the thermal gradient. Reducing the ablation duration would reduce the time thermal gradients are heating non-target sectors. However, with respect to directional MWA, a careful balance must be struck as excessive limitations on power and duration would also limit the forward ablation depth, reducing the clinical usefulness. Concerning the present invention, a power-switching or power-pulsing protocol could be used to switch the ablation applicator from treatment delivery to sensor mode for the purpose of measuring the transmission coefficient, for the purpose of improving the directivity ratio of the treatment zone, or a combination of both purposes.

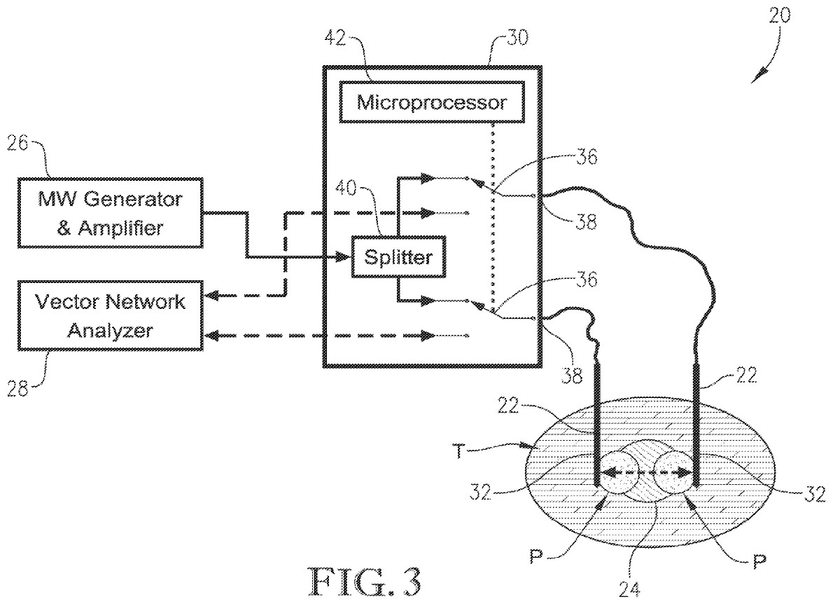

Turning to FIG. 3, one embodiment of the present invention comprises a microwave ablation system 20 that utilizes a two-channel directional applicator configuration for ablation of tissue T. The system 20 preferably includes directional applicators 22 placed at the periphery and on opposite sides of a target tissue section 24 that is targeted for thermal ablation, such as a tumor. In the illustrated embodiment, the system 20 also preferably includes a clinical microwave generator 26, a vector network analyzer 28, and a switching device 30.

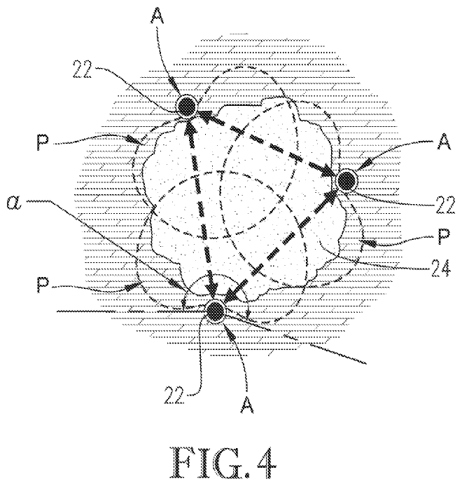

Applicators 22 each preferably include a tubular applicator body 32 and an antenna (not shown) secured within the body 32. The depicted embodiments show the use of two (2) applicators 22 (see FIG. 3) or three (3) applicators 22 (see FIG. 4) to “bracket” or surround the target tissue section 24. It will also be appreciated that more than three (3) applicators may be inserted about the target tissue section, within the ambit of the present invention.

Each applicator 22 defines a longitudinal applicator axis A and an angular radiation pattern P defined about the applicator axis A (see FIG. 4). The angular radiation pattern P defines a radiation angle α and is preferably configured to extend from the directional applicator toward the target tissue section 24 (see FIG. 4). Additional details of preferred applicator embodiments are disclosed in U.S. Publication No. 2017/0265940 and U.S. Publication No. 2020/0367966, each of which is hereby incorporated in its entirety by reference herein.

Applicators 22 are inserted into the tissue T and located in and/or adjacent the target tissue section 24. In the depicted embodiment, applicators 22 are preferably located adjacent the target tissue section 24. However, in other embodiments, it will be appreciated that one or more applicators may be located within the target tissue section. For instance, one applicator (such as an omni-directional applicator) may be placed in the center of the target tissue section, while one or more other applicators (such as a directional applicator) may be located outside of the target tissue section, along the perimeter thereof. Again, the angular radiation pattern P is preferably configured to extend from the directional applicator 22 toward the target tissue section 24. The illustrated applicators 22 are positioned at locations spaced about the target tissue section 24. Thus, the applicators 22 of the depicted embodiment are oriented to face radially inwardly relative to the center of the target tissue section 24 for “outside-in” ablation of the target tissue section 24.

Preferably, applicators 22 are oriented so that each angular radiation pattern extends from the respective applicator 22 toward at least another applicator 22. In the illustrated embodiment, applicators 22 are oriented so that the angular radiation patterns directly face each other and pass directly through the target tissue section 24.

Although each applicator 22 is preferably a directional applicator, for at least some aspects of the present invention, at least one applicator may comprise an omni-directional applicator. For instance, when inserting two (2) applicators for tissue ablation, one applicator may be directional and the other applicator may be omni-directional. When positioning more than two (2) applicators for tissue ablation, at least one of the applicators 22 is preferably directional. More preferably, all of the applicators 22 are directional applicators. However, for certain aspects of the present invention, an alternative system may include applicators that are all omni-directional.

The illustrated applicators 22 are typically placed via a percutaneous approach aided by image guidance, such as X-ray computed tomography or ultrasound. Other surgical approaches, such as laparoscopic, endoscopic, or open surgery and other visualization techniques such as MRI or direct visualization may also be used to place and orient the applicators 22.

The switching device 30 preferably comprises a switch network that includes a pair of switches 36, which are associated with applicator channels 38, a splitter 40, and the vector network analyzer 28. The switching device 30 also preferably includes a processor 42 for controlling the position of each switch 36. The splitter 40 is operably coupled to the microwave generator 26 and is operably coupled to each of the switches 36 for delivering microwave power from the generator 26. Each switch 36 is also operably coupled to the vector network analyzer 28. Vector network analyzer 28 is configured to operate as a metering device to measure a transmission coefficient.

The applicator channels 38 may be connected to a microwave transmission line that connects to the switching device 30, which allows alignment of the ablation applicator 22 to a microwave power source (such as the microwave generator 26) or the vector network analyzer 28. It is within the scope of the invention that vector network analyzer 28 may be replaced with circuits comprised of microwave sources and directional couplers, among other components, capable of obtaining a transmission coefficient between two or more ports.

The processor 42 is operably coupled to the switches 36 for selectively locating the applicator channels 38 in electrical communication with the microwave generator 26 or the vector network analyzer 28. The processor 42 preferably comprises a microcontroller. Additionally or alternatively, the processor may include microcontrollers, digital signal processors (DSPs), field-programmable gate arrays (FPGAs), analog and/or digital application-specific integrated circuits (ASICs), and the like, or combinations thereof. The processor may generally execute, process, or run instructions, code, software, firmware, programs, applications, apps, or the like, or may step through states of a finite-state machine.

With the switching device 30 first configured to align both applicator channels 38 to the vector network analyzer 28, the vector network analyzer 28 will transmit a low-intensity (sub-therapeutic), short duration broadband signal through one applicator channel that will be received by vector network analyzer through the other applicator channel and analyzed to calculate the complex transmission coefficient s₂₁. The system 20 is configured to operate in a sensing cycle, during which time the applicator channels 38 are aligned to the vector network analyzer 28.

The sensing cycle includes the step of using one or more of the applicators 28 to take a measurement associated with a dielectric property of the tissue T. That is, a measurement may be taken by only one applicator 28 or may be taken using multiple applicators 28. Preferably, the measurement comprises a transmission coefficient. However, for at least some aspects of the present invention, the applicators may be configured to measure a different parameter associated with the tissue T. As will be discussed below, the system may include one or more sensors in addition to the applicators. For instance, the system may include one or more temperature sensors and/or one or more tissue impedance sensors. During the sensing cycle, the system 20 may determine a dielectric property of the tissue (e.g., relative permittivity and/or conductivity) based upon the measurement. Prior to tissue ablation, an initial sensing cycle is used to provide a baseline transmission coefficient for a specific tumor type/biology in a specific patient.

The system 20 is then configured to operate in an ablation cycle, during which time the switching device 30 is set to align the applicator channels 38 to the microwave generator 26, so that at least one of the applicators 22 is activated and emits electromagnetic radiation that is sufficiently strong to cause ablation of the target tissue section 24. The system 20 preferably applies ablative power to the target tissue section 24 for a predefined period (e.g., for one second, thirty seconds, one minute, two minutes, etc.). In the depicted embodiment, microwave power may be supplied from a single microwave source (such as microwave generator 26) to multiple applicators 22 via the splitter 40 (or from multiple independent microwave sources, as will be shown in a subsequent embodiment), so that each applicator 22 is activated to emit electromagnetic radiation for causing ablation of the target tissue section 24.

Once the first ablation cycle has been terminated, the switching device 30 will realign the applicator channels 38 to the vector network analyzer 28, which will repeat the broadband transmission coefficient measurement between applicators 22 in a second sensing cycle. The second sensing cycle transmission coefficient measurement will be compared to the transmission coefficient measured during the baseline sensing cycle and analyzed for indications of complete target tumor ablation.

Alternatively, the comparison can be between ATC values, which as discussed above, can be calculated by averaging the magnitude of the transmission coefficient at multiple frequencies and normalized to a baseline transmission coefficient. The ATC values compared can be the ATC value from the current sensing cycle and the ATC value from the immediately prior sensing cycle, or the ATC value from the current sensing cycle and a baseline ATC value. Completeness of tissue ablation can be determined from an observation of a trend in the slowing down of the magnitude of the decrease between adjacent ATC values, or the reaching of a predetermined change in magnitude from the ATC value versus the baseline ATC value. Similarly, the comparison can be between GD values, which is the derivative of the phase of the transmission coefficient, with similar trends as to ATC values being used to indicate completeness of tissue ablation.

Without the proper indications of complete ablation, or without elapse of a maximum procedure duration setpoint safety, the ablation and sensing cycles will continue to alternate. That is, the system 20 is preferably configured to perform one or more additional treatment iterations that each include an ablation cycle and a sensing cycle that follows the ablation cycle.

It is understood that various time periods may be used for the ablation cycle such as 1 s, 30 s, 2 min, etc., and that the duration between sense/treat cycle may be adjusted during the course of the ablation. For example, during the initial phase of the procedure when the ablation zone is unlikely to have extended to the treatment margins, the interval between sensing cycles could be quite long. This interval could be considerably shortened as the ablation progresses and approaches the duration when the treatment margin is anticipated to approach the target boundary. However, if the ablation cycle is too short in duration between sensing cycles, or if the sensing cycle is too long relative to the ablation cycle, then blood perfusion of the surrounding tissue may cool the target tissue sufficiently to limit the maximum ablation zone achievable. Furthermore, if the ablation cycle is too long in duration, then the system may not have enough time resolution to provide a clinically actionable indication of the time complete ablation is achieved. The optimal ablation and sensing cycle lengths may be impacted by the specific ablation applicator design, the applicator separation distance, the target tissue size, the target tissue type, and the applied power level among other case-specific variables.

The transmission coefficient measurements may be taken only from the first applicator to the second applicator (s₂₁), only from the second applicator to the first applicator (s₁₂), or from both applicators during any particular sensing cycle. To achieve larger composite ablation zones and treat larger tumors, more than two applicators may be used to bracket and ablate the larger tumor. For this use case, the transmission coefficient may be measured between any or all combination of pairs of applicators. As shown in FIG. 4, measuring the transmission coefficient between multiple pairs of ablation applicators surrounding the target tissue may give increased resolution of the ablation status of the periphery of the tumor relative to a single pair of ablation applicators.

Changes in the complex valued transmission coefficient, or calculated ATC and/or GD values, may then be used to indicate complete ablation between the applicators. Analysis could be restricted to the magnitude, phase, or both magnitude and phase of the measured transmission coefficient data. Analysis techniques could include a model-based approach where knowledge of the anticipated radiation pattern and geometry of the space between the two antennas is used to construct a parameterized model of the transmission channel between the two applicators. Estimation of the model parameters from the measured s₂₁ data could then be used to provide an estimate of treatment extent. While this approach is well grounded in theory, the number of unknown model parameters on a patient-specific basis may be large. Alternatively, empirically based approaches could be used where changes in certain features of the transmission coefficient spectrum could be benchmarked against ablation extent. Benchmarking data could be gathered from benchtop experiments, experiments in animals in vivo, or collated from prior clinical procedures. Yet another approach could include the use of artificial intelligence-based algorithms, trained with experimental and/or clinical data, to determine whether a candidate change in transmission coefficient is indicative of through ablation between the applicators.

In a preferred embodiment, complete ablation of the target could be indicated by averaging the difference of data points between the current and prior cycle transmission coefficient measurements as shown in FIGS. 5A and 5B. The photographs of FIGS. 5C-5F show how ablation progresses over time. The transmission coefficient data sets may be preprocessed using a variety of techniques such as digital filtering. In addition, the transmission coefficient data sets may be used to calculate ATC and/or GD. Should the average difference between the current and prior cycle data sets be sufficiently small, for example less than 5%, then the intervening target tissue between ablation applicators has been sufficiently dehydrated and desiccated and is no longer being rapidly modified by the ablation process. The ablation procedure should be terminated to minimize excessive thermal conduction to surrounding tissue, causing undesired damage to healthy structures. The average difference between the current and prior cycle transmission coefficients, ATC, or GD for indicating sufficient ablation (such that the ablation procedure has been completed) preferably ranges from about 0% to about 10% and, more preferably, ranges from about 0% to about 5%. However, for at least certain aspects of the present invention, the average difference between the current and prior cycle transmission coefficient, ATC, or GD for indicating sufficient ablation may fall outside of these ranges.

Conversely, if the average difference between current and prior cycle data sets is sufficiently large, for example greater than 5%, then the intervening tissue is still being rapidly modified by the ablation process and the ablation procedure should continue to ensure complete ablation of the intervening target tissue. The average difference between the current and prior cycle transmission coefficients, ATC, or GD for indicating insufficient ablation (such that the ablation procedure should continue) preferably ranges from about 2% to about 100% and, more preferably, ranges from about 5% to about 100%. Hereafter, indication of a sufficiently small difference between the current and prior cycle data sets will be defined as transmission coefficient, ATC, or GD stabilization. It is within the scope of the current invention that other percentage changes may be used to establish transmission coefficient, ATC, or GD stabilization. Transmission coefficient, ATC, or GD stabilization may be established using data from a broadband measurement, a narrowband measurement, multiple discrete frequency measurements, or single frequency measurement of the transmission coefficient between a pair of applicators.

Additional indications may also be used independently or in combination with the transmission coefficient, ATC, or GD stabilization method to determine complete ablation. For example, a setpoint of a greater than 30% change between the current time transmission coefficient measurement or calculated ATC or GD compared to the baseline transmission coefficient measurement or baseline ATC or GD may also be required to indicate complete ablation has been achieved. The average difference between the current transmission coefficient, ATC, or GD and the baseline transmission coefficient, ATC, or GD for indicating complete ablation preferably ranges from about 20% to about 100% and, more preferably, ranges from about 30% to about 100%. If used in combination with the transmission coefficient, ATC, or GD stabilization method, then the presence of both indications would provide extra reliability in indicating complete ablation. Alternatively, a maximum change setpoint may provide a safety mechanism to prevent excessive ablation of surrounding tissues. For example, if the current cycle transmission coefficient measurement, or calculated ATC or GD exceeds a 40% difference from the baseline measurement, then there is increased risk of thermal damage to surrounding tissue at the periphery of the targeted ablation zone and the ablation procedure should be terminated immediately. Alternatively, a minimum change setpoint may be used to indicate an unforeseen issue potentially impacting the success of the ablation. For example, if the current time transmission coefficient measurement or calculated ATC or GD has not changed by more than 10% by halfway through the procedure, then perhaps some unforeseen complication, such as an unexpectedly vascular target tissue, is influencing the growth of the ablation zone and more extensive post-procedure analysis and possible re-treatment should be considered. It is within the scope of the current invention that other percentage changes may be used to establish the minimum or maximum change setpoints.

In the preferred embodiment, the switching device 30 (including the controller 42) is contained in a self-contained module that can be connected in-line via microwave transmission cables between the clinical microwave generator 26 and the ablation applicators 22. Once the controller 42 of the current invention has determined complete ablation of the target has been achieved, the system 20 may initiate automatic termination of the ablation procedure by sending an electronic signal to the microwave generator 26 to stop power output. Further, the controller 42 may automatically operate the switching device 30 to switch the system 20 from the ablation cycle back to the sensing cycle. Alternatively, a separate switch (not shown) within the self-contained module may open to isolate the microwave power source from the ablation applicators 22. In another alternative, the present invention may be configured as an operator aid where indication of complete ablation is provided through an indicator light or buzzer and the final termination of the procedure is accomplished by the operator manually turning off the microwave power source. It is within the scope of the present invention for the switching hardware and control system to be integrated within a clinical microwave generator enclosure, with the switching hardware, control system, and microwave generator sharing at least some hardware and/or software components.

Switching between ablation and sensing cycles also provides a benefit of improving the directivity of DMWA applicators. During the ablation cycle, microwave energy may be radiated and deposited in the tissue region dictated by the design of the directional antenna. This region would rapidly heat, modifying the dielectric and thermal properties of the tissue through desiccation and dehydration; at 55-60° C. the tissue would coagulate, shutting off the heat sink effect of blood perfusion. During the sensing portion of the cycle, excessive rise of the central peak temperature would be halted; blood perfusion in the surrounding tissue would cool the ablation zone margin, particularly in the non-target sectors to the side and behind the applicator. When the ablation cycle returns, the microwave energy would be absorbed incrementally less in the previously heated and partially desiccated tissue and radiate incrementally further in the forward direction. This could shift the thermal “hot spot” of the ablation more toward the desired direction and further from the undesired direction, reducing thermal gradients driving heat transfer to the non-target sectors and improving ablation directivity.

A directivity ratio approaching infinity (zero backward heating) would be of high importance for clinical users who are concerned with precision control of the treatment zone and avoidance of thermal damage to non-target nearby critical anatomy. As such, a user interested in highly directional treatment of a relatively small target may use a single DMWA applicator that replaces the sensing cycle with an “off cycle” to improve the directivity of the ablation zone.

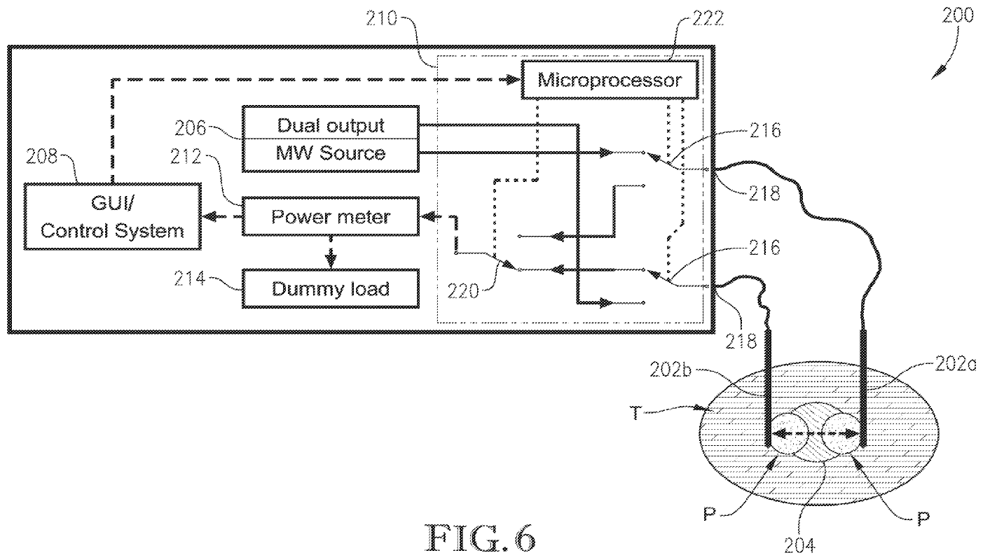

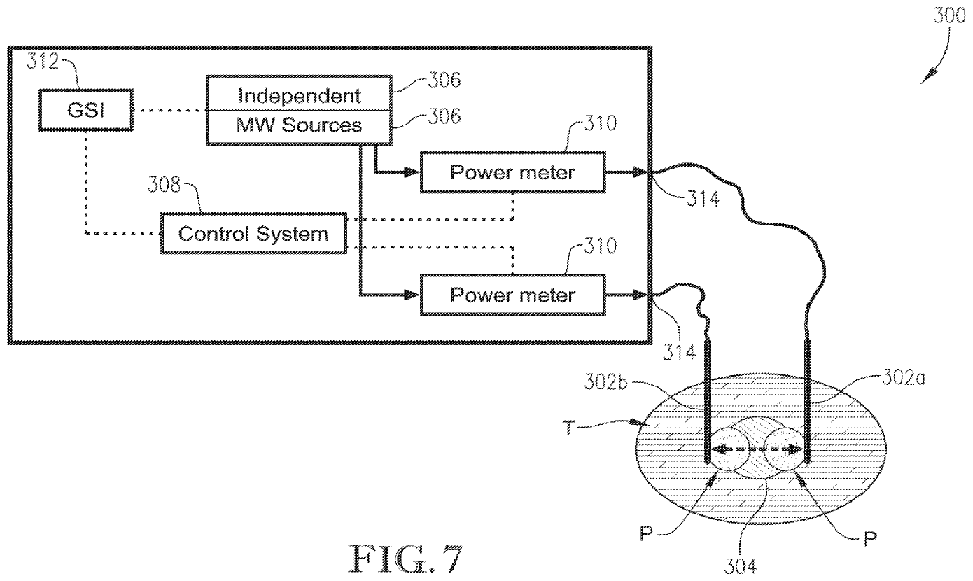

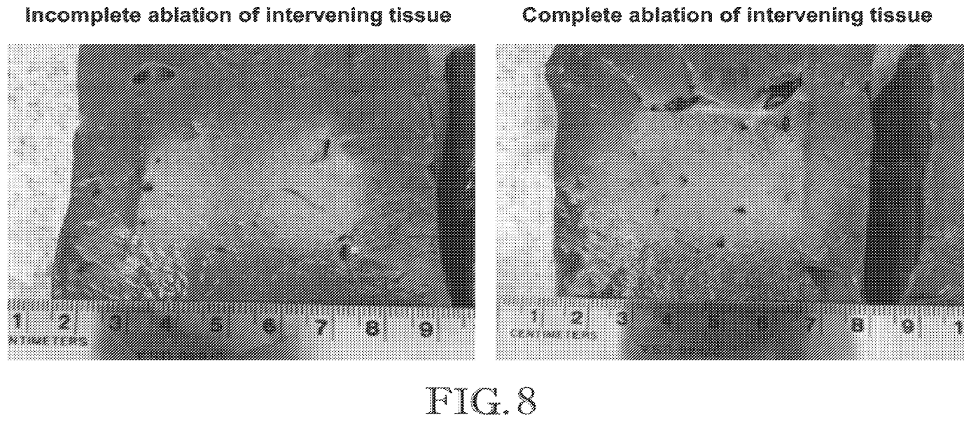

Turning to FIGS. 6 and 7, alternative preferred embodiments of the present invention are depicted. For the sake of brevity, the remaining description will focus primarily on the differences of these alternative embodiments from the preferred embodiment described above.

Referring initially to FIG. 6, an alternative microwave ablation system 200 preferably includes first and second directional applicators 202a,b placed at the periphery and on opposite sides of a target tissue section 204 that is targeted for thermal ablation. In the illustrated embodiment, the system 200 also preferably includes a dual-output microwave power source 206, a control system 208, a switching device 210, a power meter 212, and a dummy load 214. The control system 208 also preferably includes a graphical user interface (GUI).

The switching device 210 preferably comprises a switch network that includes a pair of applicator switches 216, which are associated with applicator channels 218, and a metering switch 220. The switching device 210 also preferably includes a microprocessor 222 for controlling the position of each of the switches 216 and 220. The metering switch 220 is operably coupled to the power meter 212 and is operably coupled to each of the applicator switches 216 for permitting the power meter 212 to measure the power received by one antenna 202 (such as antenna 202b in the depicted configuration) from the other antenna 202 (such as antenna 202a in the depicted configuration). Such measurements may be used to determine the transmission coefficient. The applicator switches 216 are operably coupled to the dual-output microwave power source 206 and to each of the applicators 202 for delivering microwave power from the microwave power source 206.

The applicator channels 218 may be connected to a microwave transmission line that connects to the switching device 210, which allows alignment of the ablation applicators 202 to the microwave power source 206 or the power meter 212. It is within the scope of the invention that power meter 212 may be replaced with circuits comprised of microwave power sources and directional couplers, among other components, capable of measuring microwave signal level.

The microprocessor 222 is operably coupled to the switches 216 and 220 for selectively locating the applicator channels 218 in electrical communication with the microwave power source 206 or the power meter 212.

The alternative system 200 replaces the vector network analyzer 28 of the previous embodiment with a power meter 212 connected in-line with a matched dummy load 214. In this case, a single microwave power source through the inclusion of a splitter or multiple independent microwave power sources supply microwave power to the ablation applicator channels 218 through microwave transmission cables 224 and an in-line switching device 210. While one applicator switch 216 is aligned so that one applicator channel 218 receives microwave power, the other applicator switch 216 is aligned so that the other applicator channel 218 is in communication with the power meter 212.

A portion of the microwave power being radiated by the first applicator 202a is received by the second applicator 202b and sent to the power meter 212. The initial power transmission value can be recorded at the onset of the ablation procedure to establish the procedure-specific power transmission baseline. The power transmission from the first applicator 202a to the second applicator 202b can then be monitored continuously. As the power transmission between the applicator changes sufficiently from the baseline measurement, the system 20 can then provide the clinical user an automatic action or other indication that ablation of the target tissue is complete.

During an ablation, the applicator channels 218 periodically alternate between alignment to the microwave power source 206 (for conducting an ablation cycle) and to the power meter 212 (for conducting a sensing cycle) to provide uniform, multi-directional, outside-in heating of the target tissue. It is within the scope of the invention that both applicators 202a,b may be aligned to receive microwave power at the same time, affording a faster ablation of the target tissue, with one, or multiple of the ablation applicators 202 periodically being realigned to the power meter 212 for measurement of the power transmission. In this embodiment, the microwave power source 206 provides a larger magnitude signal to be received and analyzed relative to sub-therapeutic output of the vector network analyzer 28 of the previous embodiment. This increase in magnitude of the signal measured may provide higher resolution of the changes in tissue status indicating complete ablation of the target tissue. However, this embodiment would likely provide information at only a single frequency, rather than a multiple frequency or broadband measurement provided by the vector network analyzer 28. Of note, it is within the scope of the current invention that the microwave power source 206 may instead operate over a narrow frequency band centered on a specified frequency, preferably one in the ISM band.

Turning to FIG. 7, in another embodiment, an alternative microwave ablation system 300 preferably includes first and second directional applicators 302a,b placed at the periphery and on opposite sides of a target tissue section 304 that is targeted for thermal ablation. In the illustrated embodiment, the system 300 also preferably includes independent microwave power sources 306, a control system 308, power meters 310, and a graphical user interface (GUI) 312.

The power meters 310 are in communication with respective microwave power sources 306. Power meters 310 are also in communication with respective applicator channels 314. The control system 308 is operably coupled to the GUI 312 and the power meters 310 for selectively controlling the power meters 310. Although two (2) applicators 302 are depicted, it will be understood that the microwave ablation system may include more than two applicators.

The microwave power sources 306, each operating at slightly different microwave frequencies, are used to power multiple ablation applicator channels 314. During microwave ablation, power from one microwave power source 306 is radiated from the connected applicator 302, through the target tissue, and is received by the other applicator(s) 302. Connected in-line with each microwave applicator channel 314 is a power meter 310 configured to read the power received from each of the specific frequencies of the other applicator channel(s) 314. The recorded changes in power transmission levels from each pair of applicators 302 can then be analyzed for indications of complete tissue ablation. This embodiment offers the advantage of continuous ablation (i.e., no need for switching alignment of the applicators 302 between a microwave power source 306 and a power meter 310), allowing for a faster procedure, while also providing continuous indication of the target tissue status, providing higher time resolution of procedure complete. However, this embodiment would rely on custom engineering of sub-assemblies such as the independent microwave power sources and power meters as the would need to be optimized for slightly different frequencies.

Another embodiment could include use of an independent broadband signal source, transmitting a broadband pulse from one of the antennas. A directional coupler is used to assess a portion of the signal on the transmitting terminal, and likewise on the receiving terminal. Each of the directional couplers is connected to a sensor for measuring magnitude and phase signals, which would be used for determining complex valued transmission coefficient. The analysis of the transmitted and received signals may occur in either the time domain or the frequency domain, with the appropriate use of Fourier techniques.

Clinical effectiveness of the present invention may be increased if used in combination with additional temperature or tissue impedance sensors. For example, since the bracketing ablation technique heats from the outside in, placing a temperature sensor in the center of the target tumor could provide additional confirmation that the core of the target has reached ablative temperatures. It is within the scope of the present invention that this temperature data could be used independently or in combination with transmission coefficient, ATC, GD, or power transmission data to provide indication of complete ablation. Another example would be placement of a temperature sensor nearby a critical sensitive structure where the ablation procedure could be terminated early if that temperature sensor indicates temperatures rising to damaging levels. Using tissue impedance independently or in combination with transmission coefficient, ATC, GD, or power transmission data to provide indication of complete ablation can provide an additional data set to more reliable confirm complete ablation of the target tissue. However, minimizing the number of needles and devices that must be placed in a patient during a minimally invasive procedure is critical to reducing the duration of the procedure, the potential need for local vs. general anesthesia, and the patient's post-procedure pain level and recovery time. As such, a major clinical advantage of the previously described embodiments of current invention is that the treatment devices serve a shared purpose as monitoring devices and additional devices are not required to inserted during the procedure.

Since the magnitude of the signal transmitted between applicators 22,202,302 will be maximum when the applicators 22,202,302 are directly facing each other and are in-line, the current invention can also be used to position and confirm orientation of the ablation applicators 22,202,302 pre-procedure. To do so, the system would configure the applicator channels to be aligned to the vector network analyzer 28 or the microwave power source 206,306 set to output a sub-therapeutic power level associated with a sensing cycle. In this system configuration, the clinical user would slowly rotate one or more applicators 22,202,302 about the applicator axis A based upon the measured transmission coefficient or calculated ATC or GD, then slowly insert or retract one or more applicators along the applicator axis A based upon the measured transmission coefficient or calculated ATC or GD, until they observe a maximum signal transmission level on the system display.

As different tissue and tumor types have different dielectric properties, which impact the ablation outcome but are exceedingly difficult to determine pre-procedure, the current invention also offers the opportunity to use the baseline transmission coefficient, calculated ATC or GD, or power transmission themselves as part of the treatment planning process. For example, should a clinical user place and orient the ablation applicators as previously described, and upon taking a baseline measurement determine that the target tissue is less conductive than expected, they may then have the ability to decide to modify the ablation power and/or duration given this new information. It is also anticipated that baseline transmission coefficient measurements or calculate ATC or GD across a number of patients may be applied to determine the relationship between baseline transmission coefficients, ATC, or GD and dielectric properties of a given patient's tumor.

As used herein, the phrase “and/or,” when used in a list of two or more items, means that any one of the listed items can be employed by itself or any combination of two or more of the listed items can be employed. For example, if an assembly is described as containing elements A, B, and/or C, the assembly may contain A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

As used herein, the term “includes” may refer to an item that includes something as a part thereof or is entirely made up of that something.

Although the above description presents features of preferred embodiments of the present invention, other preferred embodiments may also be created in keeping with the principles of the invention. Such other preferred embodiments may, for instance, be provided with features drawn from one or more of the embodiments described above. Yet further, such other preferred embodiments may include features from multiple embodiments described above, particularly where such features are compatible for use together despite having been presented independently as part of separate embodiments in the above description.

The preferred forms of the invention described above are to be used as illustration only, and should not be utilized in a limiting sense in interpreting the scope of the present invention. Obvious modifications to the exemplary embodiments, as hereinabove set forth, could be readily made by those skilled in the art without departing from the spirit of the present invention.

The inventors hereby state their intent to rely on the Doctrine of Equivalents to determine and assess the reasonably fair scope of the present invention as pertains to any apparatus not materially departing from but outside the literal scope of the invention as set forth in the following claims.

1. A method of ablating tissue using a microwave ablation system comprising a plurality of applicators, said method comprising the steps of:
(a) having the applicators inserted into the tissue and located in and/or adjacent a target tissue section;
(b) having the system perform an ablation cycle by activating at least one of the applicators so that the at least one applicator emits electromagnetic radiation that is sufficiently strong to cause ablation of the target tissue section,
said at least one applicator comprising a directional applicator that emits radiation to define an angular radiation pattern,
step (a) including the step of orienting the directional applicator so that the angular radiation pattern extends from the directional applicator toward the target tissue section; and
(c) having the system perform a sensing cycle that includes the step of using one or more of the applicators to take a measurement associated with a dielectric property of the tissue.

2. (canceled)

3. The method as claimed in claim 1,
step (b) including the step of activating multiple ones of the applicators so that the multiple applicators each emit electromagnetic radiation for causing ablation of the target tissue section,
each of said applicators comprising a directional applicator that emits radiation to define the angular radiation pattern,
step (a) including the step of orienting the directional applicators so that each angular radiation pattern extends from the respective directional applicator toward at least another one of the directional applicators.

4. (canceled)

5. (canceled)

6. The method as claimed in claim 1,
step (a) including the step of orienting the directional applicator based upon the measurement to maximize a signal transmission level between the applicators,
said orienting step including (i) the step of rotating the directional applicator relative to the tissue about an applicator axis, and/or (ii) the step of inserting or retracting the directional applicator relative to the tissue along the applicator axis.

7. (canceled)

8. (canceled)

9. The method as claimed in claim 1, further comprising the step of:
(d) having the system switch itself between step (b) and step (c) to have the ablation and sensing cycles performed at different times, said microwave ablation system including a switching device that performs step (d).

10. (canceled)

11. The method as claimed in claim 1, further comprising the step of:
(d) having the system perform step (b) and step (c) simultaneously, with one of the applicators being activated to emit electromagnetic radiation for causing ablation of the target tissue section, and another one of the applicators being used to take the measurement.

12. The method as claimed in claim 1,
step (b) including the step of activating multiple ones of the applicators so that the multiple applicators each emit electromagnetic radiation for causing ablation of the target tissue section,
each of said applicators comprising a directional applicator that emits radiation to define the angular radiation pattern,
step (a) including the step of orienting the directional applicators so that each angular radiation pattern extends from the respective directional applicator toward at least another one of the directional applicators.

13. (canceled)

14. (canceled)

15. The method as claimed in claim 1,
step (c), wherein the measurement comprises a transmission coefficient of the tissue.

16. The method as claimed in claim 15,
step (c) including the step of determining the tissue dielectric property based upon the measurement.

17. The method as claimed in claim 15,
step (a) including the step of orienting the directional applicator based upon the measured transmission coefficient.

18. The method as claimed in claim 1,
step (c) being performed prior to step (b) in order to sense a baseline measurement.

19. (canceled)

20. The method as claimed in claim 18,
step (c), wherein the baseline measurement comprises a baseline transmission coefficient of the tissue.

21. The method as claimed in claim 18, further comprising the steps of:
(d) having the system perform another sensing cycle after step (b) so that the ablation cycle and the another sensing cycle cooperatively provide a treatment iteration, with the another sensing cycle including the step of using the at least one applicator to take another measurement associated with the dielectric property of the tissue; and
(e) having the system switch itself from step (b) to step (d).

22. (canceled)

23. The method as claimed in claim 21,
step (c), wherein the baseline measurement comprises a baseline transmission coefficient of the tissue.

24. The method as claimed in claim 23,
step (d), wherein the another measurement comprises an ablated transmission coefficient of the tissue, and
further comprising the step of:
(e) comparing the ablated transmission coefficient and the baseline transmission coefficient.

25. (canceled)

26. The method as claimed in claim 24,
step (c), further including calculating a baseline average transmission coefficient by averaging a magnitude of the baseline transmission coefficient at multiple frequencies;
step (d), further including calculating an ablated average transmission coefficient by averaging a magnitude of the ablated transmission coefficient at multiple frequencies; and further comprising the step of
(e) comparing the baseline average transmission coefficient and the ablated average transmission coefficient.

27. The method as claimed in claim 24,
step (d), wherein the ablated average transmission coefficient is normalized to the baseline average transmission coefficient.

28. The method as claimed in claim 24,
step (c), further including calculating a baseline group delay by taking the derivative of a phase of the baseline transmission coefficient;
step (d), further including calculating an ablated group delay by taking the derivative of a phase of the ablated transmission coefficient, and wherein the ablated group delay is normalized to the baseline group delay; and further comprising the step of
(e) comparing the baseline group delay and the ablated group delay.

29. (canceled)

30. The method as claimed in claim 25, further comprising the step of:
(f) terminating the ablation procedure based upon the comparison of step (e).

31. The method as claimed in claim 25, further comprising the steps of:
(f) after step (e), having the system perform one or more additional treatment iterations that each include an ablation cycle and a sensing cycle that follows the ablation cycle, with each sensing cycle of the one or more additional treatment iterations including the step of using one or more of the applicators to take a measurement comprising an ablated transmission coefficient of the tissue;
(g) terminating the ablation procedure if the difference between the transmission coefficient of the most recent sensing cycle and the transmission coefficient of one of the prior cycles is below a predetermined minimum change setpoint; and
(h) terminating the ablation procedure if the average difference between the transmission coefficient of the most recent sensing cycle and the baseline transmission coefficient is greater than a predetermined maximum change setpoint.

32. (canceled)

33. (canceled)

34. (canceled)

35. The method as claimed in claim 26, further comprising the steps of:
(f) after step (e), having the system perform one or more additional treatment iterations that each include an ablation cycle and a sensing cycle that follows the ablation cycle, with each sensing cycle of the one or more additional treatment iterations including the step of using one or more of the applicators to take a measurement comprising an ablated transmission coefficient of the tissue, and calculating an ablated average transmission coefficient and/or an ablated group delay;
(g) terminating the ablation procedure if the difference between the average transmission coefficient or group delay of the most recent sensing cycle and the average transmission coefficient or group delay of one of the prior cycles is below a predetermined minimum change setpoint; and
(h) terminating the ablation procedure if the average difference between the average transmission coefficient or group delay of the most recent sensing cycle and the baseline average transmission coefficient or baseline group delay is greater than a predetermined maximum change setpoint.

36. (canceled)

37. (canceled)

38. The method as claimed in claim 1,
step (c), wherein the measurement comprises a transmission coefficient of the tissue,
step (c) including the step of using the applicators to take transmission coefficient measurements for the target tissue section at a plurality of frequencies.

39. A microwave ablation system configured to ablate tissue, said microwave ablation system comprising:
at least one microwave source;
a plurality of applicators operable to be inserted into the tissue and located in and/or adjacent a target tissue section,
at least one of said applicators configured to receive power from the microwave source during an ablation cycle and emit electromagnetic radiation that is sufficiently strong to cause ablation of the target tissue section,
said at least one applicator comprising a directional applicator that emits radiation to define an angular radiation pattern, with the directional applicator configured to be oriented so that the angular radiation pattern extends from the directional applicator toward the target tissue section; and
a metering device configured to receive a signal from at least one of the applicators during a sensing cycle to take a measurement associated with a dielectric property of the tissue.

40-50. (canceled)
